(19) United States
(12) Patent Application Publication
(10) Pub. No.: US 2026/0144746 A1
(43) Pub. Date: May 28, 2026

(54) Protein Based Soft Chew Tablets

(71) Applicant: Fertin Pharma A/S, Vejle (DK)

(72) Inventors: Helle Wittorff, Vejle Ost (DK); Jane Skov Grabow Jensen, Østbirk (DK)

(73) Assignee: Fertin Pharma A/S, Vejle (DK)

(21) Appl. No.: 19/216,992

(22) Filed: May 23, 2025

(30) Foreign Application Priority Data
Nov. 25, 2024 (DK) PA202430723

(51) Int. Cl. A61K 9/00; A61K 9/20; A61K 9/28; A61K 45/06

(52) U.S. Cl. CPC A61K 9/0056; A61K 9/2009; A61K 9/2018; A61K 9/2059; A61K 9/2063; A61K 9/2068; A61K 9/288; A61K 45/06

(57) ABSTRACT

A soft chew tablet based on proteins includes a core constituting a coherent uncooked mass including: one or more indigestible water soluble fibers in an amount of 10 to 45% by weight of the core, one or more powdered proteins in an amount of 10 to 60% by weight of the core, and one or more texturizers in an amount of 0.1 to 25% by weight of the core.

### FIELD OF INVENTION

The present invention relates to soft chew tablets based on proteins, such as proteins originating from dairy proteins and plant based proteins. The soft chew tablets are particularly beneficial for administering proteins and/or dietary fibers and has potential for delivering a relatively high amount of active ingredients.

### BACKGROUND

Various attempts have been made to apply soft chew tablets used in the confectionery industry as delivery vehicles for active ingredients. While soft chew tablets are generally attractive for consumers within the confectionery segment, various issues arise if active ingredients are to be formulated into these products.

Traditionally, soft chew tablets are known to be made in a manufacturing process that involves recrystallization of sugars and other ingredients and involving extensive heat treatment during processing. Although the texture of such products becomes consumer friendly, high temperatures may be challenging to heat sensitive active ingredients. Also, melting of ingredients may impact release characteristics in the products as well as the required load level of such ingredients in the products.

Furthermore, the use of sugars may be inappropriate in various formulations and for administration of certain active ingredients, such as active ingredients with health improving properties.

Another challenge of heat treatment in soft chew tablets and especially cooking during the process is that the texture of the products becomes unsuitable for delivery of certain active ingredients, such as certain vitamins, minerals, and the like. The need for delivery of large doses of active ingredients may be counteracted by a dense and compacted structure of the soft chew tablets. Additionally, high loads of active ingredients may in itself counteract the intended texture of such products.

In recent years, variations of the manufacturing process of soft chew tablets have been suggested for active ingredients, processes that involve less heat transfer to the ingredients during the manufacturing process. However, various challenges still apply for these processes, such as undesirable texture of the products as well as a high degree of stickiness of the products to surfaces. The challenge of stickiness may be pronounced for sugar-related products.

Hence, there is a need in the prior art for improved delivery vehicles that solve the above-referenced challenges and problems of the prior art. In particular, there is a need in the prior art for new soft chew tablet delivery vehicles that support appropriate delivery of active ingredients, such as vitamins, minerals, and the like, combined with beneficial sensorial properties, including improved mouthfeel and texture.

### SUMMARY

Accordingly, in a first aspect there is provided a soft chew tablet based on proteins, the soft chew tablet comprising: a core constituting a coherent uncooked mass comprising: one or more indigestible water soluble fibers in an amount of 10 to 45% by weight of the core, one or more powdered proteins in an amount of 10 to 60% by weight of the core, and one or more texturizers in an amount of 0.1 to 25% by weight of the core.

In another aspect, there is provided a soft chew tablet based on proteins, the soft chew tablet comprising: a core constituting a coherent mass comprising: one or more indigestible water soluble fibers in an amount of 10 to 45% by weight of the core, one or more powdered proteins in an amount of 10 to 60% by weight of the core, and one or more texturizers in an amount of 0.1 to 25% by weight of the core. The embodiments and considerations described below apply to both the first and second aspect of the invention.

In the present context, the “core” is to be understood as the entire tablet, if nothing else is implied or mentioned. While the “core” may be provided with an outer coating, such as an outer “chocolate coating” or “sugar free hard coating”, the “core” may be absent any outer coating. One of the advantages of a “core” without an outer coating may be that less processing is required and more pronounced “soft chew” characteristics is present upon chewing the “soft chew tablets” absent an outer coating. Without being bound by theory, it is believed that the presence of powdered proteins instead of sugar-based components makes it possible to obtain a product without the need of an outer coating. In support, it was seen that less hardening of the product took place over time compared to soft chew tablets with sugar-based components. Additionally, the texture of the soft chew tablets according to the invention was not expected to be beneficial compared to soft chew tablets with sugars.

One of the great benefits of applying powdered proteins instead of sugar-based components is that various active ingredients that usually are not applied with sugar-based components may be applied in the format of “soft chew tablets”. Resembling “soft chew tablets” based on sugar components is a difficult task, not only in terms of manufacturing requirements, but certainly also in terms of resembling the texture and properties of sugar-based “soft chew tablets”.

However, the inventors surprisingly managed to produce soft chew tablets based on proteins partly or completely replacing sugar components, such as partly or completely replacing powdered sugar components contained in traditional soft chew tablets. This was surprisingly achieved by incorporation of various further components in the tablet working in synergy with the powdered proteins alcohol according to the invention, including a unique “texture system”. In the present context, a “texture system” or “texturizing system” is intended to include the “one or more texturizers” according to the invention.

Another advantage with the present invention is that the soft chew tablets according to the invention may be less sticky compared to a sugar version. This is a great benefit and not foreseen by the inventors. Less stickiness to the teeth and surfaces may be obtained and this also implies that an outer coating is not always necessary.

Generally, the present invention solves various other challenges of the prior art and may provide further advantages compared to the prior art in terms of improved mouthfeel, texture as well as improved delivery of proteins and dietary fibers as well as active ingredients. An important advantage is that the stickiness of the product may be reduced or even avoided by the present invention, which means that handling and convenience may be greatly improved. Another important advantage is that challenges in terms of moisture balance in the product may be greatly improved.

Additionally, it was a surprise to the inventors that a coating surrounding the core of the present invention in some instances was not always necessary to help avoiding too much hardening over time during storage. The hardening of the core during storage was regularly measured with a texture analyzer and it could be seen that hardening was not significantly pronounced without a coating. This was a surprise to the inventors.

Delivery of health-improving proteins and dietary fibers with various different health-improving advantages has traditionally involved challenging problems, particularly if a relatively high amount of these proteins and fibers are to be delivered to a person in need thereof. With the present invention, it was highly surprising that the main base for soft chew tablets (powdered sugar components) would be possible to replace partly or completely with the powdered proteins according to the invention without substantially compromising the nature of soft chew tablets. The sense of soft chew was seen to be maintained by replacing powdered sugars partly or completely with powdered proteins, such as dairy proteins and/or plant based proteins, which was not expected by the inventors.

Accordingly, the present invention can be seen as leveraging from a synergy effect of substituting powdered sugar components (that are not healthy) with powdered proteins according to the invention (that are healthy as nutrients).

In some embodiments, the soft chew tablet has a total weight of 0.5 to 5.0 g. In some embodiments, the soft chew tablet has a total weight of 1.0 to 5.0 g. In some embodiments, the soft chew tablet has a total weight of 0.5 to 4.0 g. In some embodiments, the soft chew tablet has a total weight of 0.5 to 3.0 g. In some embodiments, the soft chew tablet has a total weight of 1.5 to 4.0 g. In some embodiments, the soft chew tablet has a total weight of 2.0 to 3.0 g.

In some embodiments, when the core is applied with an outer coating, the core of the tablet has a total weight 0.5 to 4.0 g. In some embodiments, when the core is applied with an outer coating, the core of the tablet has a total weight 1.0 to 4.0 g. In some embodiments, when the core is applied with an outer coating, the core of the tablet has a total weight of 1.0 to 3.5 g. In some embodiments, when the core is applied with an outer coating, the core of the tablet has a total weight of 1.5 to 3.0 g.

In some embodiments, the coherent mass has not been re-crystallized during formation of the core, which is usually the case for sugar based soft chew tablets.

In the present context, “re-crystallized” is intended to mean that crystalline ingredients applied in the process are not melted during the process and re-crystallized during or after cooling. Typically, processes involving cooking as seen for sugar based soft chew tablets, such as by heating crystalline ingredients and other ingredients to 130-150 degree Celsius during processing, completely melts the mass of ingredients, which results in the ingredients being spun together in the mass. During or after cooling, the mass of spun ingredients re-crystallizes in such cooking process. In the present context, cooking is not invoked to the ingredients, and the mass of core of the soft chew tablets is “an uncooked mass”. On a microscopic level, the texture of a cooked mass that has been re-crystallized may be substantially different than the texture of the mass of the core in the present invention, while retaining the characteristics of a “soft chew tablet” on a macroscopic, perceived level. Such cooked mass is associated with various drawbacks, including challenges with inferior release of active ingredients, potential harm to various proteins and dietary fibers, stickiness, etc.

A person skilled in the art would understand the intentional meaning with “soft chew tablets” in the present context. By the formulation presented to the instantly claimed “soft chew tablets”, the inventors managed to resemble “soft chew tablets” made by the aforementioned cooking process, but without the challenges associated with this process. Traditionally, the term “soft chew tablets”, as would be understood by one skilled in the art, is intrinsically linked to a cooking process, thus not including other formats that are outside the category of “soft chew tablets”, such as compressed tablets. One of the great benefits of the present invention is that cooking may be avoided, while still preserving the characteristic of “soft chew tablets” in the traditional context on a macroscopic, perceived level.

The formulation provided according to the invention is associated with various advantages, such as the possibility to contain a substantial amount of active ingredients, including heat-sensitive active ingredients, which is not possible with a cooked mass for traditional soft chew tablets, as well as applying a substantial amount of proteins and dietary fibers in the product.

By resembling “soft chew tablets”, the intended meaning is that texture with chew resistance is obtained, just like for cooked “soft chew tablets”. The chew resistance is characterized by some degree of elasticity and may be comparable with “chewing gum” for the first initial chews (some degree of elasticity and chew resistance), contrary to a plastic chew without elasticity as seen for products like toffees.

However, compared to chewing gum and the first initial chews in this product, the “soft chew tablets” is absent any insoluble chewing gum base. Another non-comparable product is a fondant (a sugar mass that offers no resistance or elasticity when chewing it). Chew resistance may be measured by a “texture analyzer” known to one skilled in the art or by sensorial evaluation by a test panel.

The “soft chew tablets” according to the invention are especially beneficial due to the presence of powdered proteins and optional sugar alcohols instead of sugar-based components. The powdered proteins and optional sugar alcohols were seen to work in great synergy with the other components according to the invention.

In some embodiments, the one or more powdered proteins and optional sugar alcohols of the coherent mass has been preserved during formation of the core.

In some embodiments, the coherent mass is characterized by preserved crystallinity of the one or more optional powdered sugar alcohols during formation of the core. In the present context, “preserved” means up to 100% preserved, but there may be parts of the crystalline ingredients applied in the process that may not be preserved, such as for instance up to 5% by the crystalline ingredients.

In some embodiments, the one or more optional powdered sugar alcohols has retained at least 90% crystallinity during formation of the core, such as at least 95% crystallinity.

In some embodiments, the coherent mass is a coherent non-crystalline mass.

In some embodiments, the coherent mass is characterized by a texture resembling a soft chew tablet mass with remains of particles.

In some embodiments, the coherent uncooked mass is a coherent mass with remains of particles.

In some embodiments, the coherent uncooked mass comprises sugar and/or sugar alcohol.

In some embodiments, the coherent uncooked mass is a coherent mass with remains of sugar particles and/or sugar alcohol particles.

In the present context, “remains of particles” or “remains of sugar particles and/or sugar alcohol particles” is to be understood as preservation of a major part of the physical structure of at least a part of the raw material particles applied in the formation of the soft chew tablets. For examples, if xylitol raw material particles are applied in the formation of the soft chew tablets, a substantial part of the physical structure of these particles can be identified in the final product. Upon extrusion of low speed shear mixed ingredients, a substantial part of such xylitol particles can for example be identified in the extruded coherent mass, not necessarily as complete particles but as major fractions of complete particles. In the present context, particles that are melted or otherwise intermixed with the other ingredients of the soft chew tablets without physical remains of particles are not to be considered to be “remains of particles”.

In some embodiments, the coherent mass is characterized by a texture resembling a soft chew tablet mass with remains of particles that does not crumble or fall apart upon chewing. This may for instance be seen in traditional compressed tablets.

In some embodiments, the coherent mass is extruded. Typically, the mass is made in a batch process and formed into a rope that is cut into round-shaped pieces whereupon a coating is applied. The mass may be conveyed to an extruder during the process. As an alternative to a batch process, a continuous extrusion process may be applied. For the avoidance of doubt, compressed or pressed tablets do not fall within this category.

In some embodiments, the coherent uncooked mass is an extruded coherent mass.

In some embodiments, the coherent uncooked mass is an extruded coherent mass with remains of particles.

In some embodiments, the coherent uncooked mass is an extruded coherent mass with remains of sugar particles and/or sugar alcohol particles.

In some embodiments, the coherent mass is not compressed or pressed, such as in a tableting machine. Such compression is usually applied to particular material and gives a product that is distant from the characteristic “soft chew tablets” according to the invention. In some embodiments, the coherent mass is not molded. In some embodiments, the coherent mass is not a deposited mass. These products are distant from the characteristic “soft chew tablets” according to the invention.

In some embodiments, the coherent mass is formed without melting any content of powdered sugar alcohols in the core during formation of the core, such as with a temperature below 60 degree Celsius, such as with a temperature of 20 to 40 degree Celsius.

In some embodiments, the coherent mass is formed at a temperature below 60 degree Celsius. In some embodiments, the coherent mass is formed at a temperature of 20 to 50 degree Celsius. In some embodiments, the coherent mass is formed at a temperature of 20 to 40 degree Celsius. In some embodiments, the coherent mass is formed at a temperature of 20 to 30 degree Celsius.

In some embodiments, the coherent mass is an unspun mass of ingredients. Although the powdered proteins according to the invention may have a certain polymer chain length and spatial configuration, unlike powdered sugar based components traditionally applied for soft chew tablets, the powdered proteins according to the invention may not be spun together in the product in a resistant thread-like structure.

In some embodiments, the coherent mass is formed without high speed mixing of ingredients.

In some embodiments, the coherent mass is formed by low speed shear mixing of ingredients, such as by a Z-blade mixer which is also known as a sigma-blade mixer or a sigma-blade kneader mixer. In this process, the mass may be cooled after mixing and formed into a rope by means of extrusion. In some embodiments, the coherent mass is formed without heating, except for any heating produced by friction.

In some embodiments, the coherent mass is formed by mixing in an extruder in a continuous process. In this process, the mass may be cooled in a final zone of the extruder after mixing and directly formed into a rope by the extruder. By this process, the core will not undergo the same degree of compaction of ingredients as may be seen for pressed tablets.

In some embodiments, the one or more indigestible water soluble fibers is present in an amount of 15 to 45% by weight of the core. In some embodiments, the one or more indigestible water soluble fibers is present in an amount of 15 to 40% by weight of the core. In some embodiments, the one or more indigestible water soluble fibers is present in an amount of 20 to 40% by weight of the core. In some embodiments, the one or more indigestible water soluble fibers is present in an amount of 25 to 40% by weight of the core. In some embodiments, the one or more indigestible water soluble fibers is present in an amount of 20 to 35% by weight of the core. In some embodiments, the one or more indigestible water soluble fibers is present in an amount of 20 to 30% by weight of the core.

In some embodiments, the one or more indigestible water soluble fibers has a Dextrose Equivalent (DE) of 3 to 20. In some embodiments, the one or more indigestible water soluble fibers has a Dextrose Equivalent (DE) of 5 to 20. In some embodiments, the one or more indigestible water soluble fibers has a Dextrose Equivalent (DE) of 8 to 20. In some embodiments, the one or more indigestible water soluble fibers has a Dextrose Equivalent (DE) of 8 to 15. In some embodiments, the one or more indigestible water soluble fibers has a Dextrose Equivalent (DE) of 8 to 13. In some embodiments, the one or more indigestible water soluble fibers has a Dextrose Equivalent (DE) of 10 to 20. In some embodiments, the one or more indigestible water soluble fibers has a Dextrose Equivalent (DE) of 12 to 20. In some embodiments, the one or more indigestible water soluble fibers has a Dextrose Equivalent (DE) of 15 to 20.

Dextrose Equivalent is understood by a person skilled in the art and is applied in the present context as understood in the art. Starch has a DE of 0, while dextrose has a DE of 100.

In some embodiments, the one or more indigestible water soluble fibers comprises oligosaccharides. In some embodiments, the one or more indigestible water soluble fibers is oligosaccharides. In some embodiments, the one or more indigestible water soluble fibers comprises fructo-oligosaccharides. In some embodiments, the one or more indigestible water soluble fibers is fructo-oligosaccharides.

In some embodiments, the one or more indigestible water soluble fibers comprises indigestible carbohydrate fibers. In some embodiments, the one or more indigestible water soluble fibers is indigestible carbohydrate fibers.

In some embodiments, the one or more indigestible water soluble fibers comprises polydextrose. In some embodiments, the one or more indigestible water soluble fibers is polydextrose.

In some embodiments, the one or more indigestible water soluble fibers comprises inulin. In some embodiments, the one or more indigestible water soluble fibers is inulin. In some embodiments, the one or more indigestible water soluble fibers comprises inulin having a degree of polymerization (DP) of 2-60. In some embodiments, the one or more indigestible water soluble fibers comprises inulin having a degree of polymerization (DP) of 10-60.

In some embodiments, the one or more indigestible water soluble fibers comprises indigestible maltodextrin. In some embodiments, the one or more indigestible water soluble fibers is indigestible maltodextrin. Within the scope of the invention, indigestible maltodextrin may also be referred to as digestion resistant maltodextrin.

In some embodiments, the one or more indigestible water soluble fibers comprises polydextrose. In some embodiments, the one or more indigestible water soluble fibers is polydextrose.

In some embodiments, the one or more indigestible water soluble fibers is a mixture of two or more water soluble indigestible fibers.

In some embodiments, the one or more indigestible water soluble fibers provides cohesiveness and elasticity to the core. “Elasticity” is intended to be within the acceptable limits for “soft chew tablets” as described above. In some embodiments, the one or more indigestible water soluble fibers provides cohesiveness and chew resistance to the core. Hence, the texture may be greatly improved by use of the one or more indigestible water soluble fibers according to the invention.

To the surprise of the inventors, it was possible to avoid using solid sugar syrups in the formulation by use of the one or more indigestible water soluble fibers according to the invention. This is especially advantageous for sugar-free versions of “soft chew tablets” according to the invention in order to provide a complete sugar free product.

In some embodiments, wherein the one or more indigestible water soluble fibers comprises at most 0.5% sugars by weight of the one or more indigestible water soluble fibers.

In some embodiments, the soft chew tablet is free of added sugars.

The cohesiveness and elasticity may even be improved by avoiding the use of solid sugar syrups according to the invention. In the present context, “cohesiveness” or “coherent” is intended to mean the way the mass of the core behaves when it is for instance pulled apart. A high “cohesiveness” requires a higher force to pull the core apart. In the present context, “elasticity” is intended to mean the way the mass of the core behaves when it is for instance pulled apart. A high “elasticity” is understood in the same way as when a chewing gum is pulled apart, i.e., how the material bounces back upon chewing. In this context “elasticity” may sometimes also be referred to as “chewing resistance”.

In some embodiments, the one or more indigestible water soluble fibers provides reduced stickiness to the core. It was surprisingly seen that the one or more indigestible water soluble fibers may impact the stickiness of the core. This was not anticipated by the inventors, and greatly enhances the product, especially upon chewing the product, such as by sticking to the teeth during chewing. The formulation provided according to the invention serves to achieve this property.

In some embodiments, the one or more solid sugar syrups is present in an amount up to 15% by weight of the core. In some embodiments, the one or more solid sugar syrups is present in an amount up to 10% by weight of the core. In some embodiments, the one or more solid sugar syrups is present in an amount up to 8% by weight of the core. In some embodiments, the one or more solid sugar syrups is present in an amount up to 5% by weight of the core. In some embodiments, the one or more solid sugar syrups is present in an amount up to 3% by weight of the core. In some embodiments, the one or more solid sugar syrups is present in an amount up to 2% by weight of the core. In some embodiments, the one or more solid sugar syrups is present in an amount up to 1% by weight of the core.

In some embodiments, the one or more solid sugar syrups is formed by first making a syrup, such as glucose syrup, followed by evaporation of water. In some embodiments, the one or more solid sugar syrups contain less than 6% by weight of water. “Solid sugar syrups” may sometimes also be referred to as “dried sugar syrups”

In some embodiments, the one or more solid sugar syrups has a Dextrose Equivalent (DE) of above 20. In some embodiments, the one or more solid sugar syrups has a Dextrose Equivalent (DE) of above 30.

In some embodiments, the one or more solid sugar syrups has a Dextrose Equivalent (DE) of 20 to 60. In some embodiments, the one or more solid sugar syrups has a Dextrose Equivalent (DE) of 30 to 50, In some embodiments, the one or more solid sugar syrups has a Dextrose Equivalent (DE) of 35 to 40, In some embodiments, the one or more solid sugar syrups comprises one or more solid corn syrups.

In some embodiments, the one or more solid sugar syrups provides binding properties to the mass of the core.

In some embodiments, the one or more solid sugar syrups provides stickiness to the mass of the core, which is not beneficial in absence of a coating. In some embodiments of the invention, stickiness is not preferred.

In some embodiments, the core does not comprise the one or more solid sugar syrups. This is presently preferred.

In some embodiments, the core comprises the one or more solid sugar syrups in a range of 0 to 20% by weight of the core. In some embodiments, the core comprises the one or more solid sugar syrups in a range of 0 to 10% by weight of the core. In some embodiments, the core comprises the one or more solid sugar syrups in a range of 0 to 5% by weight of the core. In some embodiments, the core comprises the one or more solid sugar syrups in a range of 0 to 3% by weight of the core. In some embodiments, the core comprises the one or more solid sugar syrups in a range of 0 to 2% by weight of the core. In some embodiments, the core comprises the one or more solid sugar syrups in a range of 0 to 1% by weight of the core.

In some embodiments, the core further comprises one or more liquid or semi-liquid sugar alcohol syrups or sugar syrups. In the present context, these are intended to also include sugar alcohols liquified by water, such as liquid sorbitol and liquid maltitol.

In some embodiments, the core further comprises one or more liquid or semi-liquid sugar alcohol syrups or sugar syrups in an amount of 0.1 to 30% by weight of the core. In some embodiments, the core further comprises one or more liquid or semi-liquid sugar alcohol syrups or sugar syrups in an amount of 0.1 to 25% by weight of the core. In some embodiments, the core further comprises one or more liquid or semi-liquid sugar alcohol syrups or sugar syrups in an amount of 0.1 to 20% by weight of the core. In some embodiments, the core further comprises one or more liquid or semi-liquid sugar alcohol syrups or sugar syrups in an amount of 1 to 20% by weight of the core. In some embodiments, the core further comprises one or more liquid or semi-liquid sugar alcohol syrups or sugar syrups in an amount of 1 to 10% by weight of the core.

In some embodiments, the core further comprises one or more liquid or semi-liquid sugar alcohol syrups in an amount of at most 10% by weight of the core. In some embodiments, the core further comprises one or more liquid or semi-liquid sugar alcohol syrups in an amount of at most 8% by weight of the core. In some embodiments, the core further comprises one or more liquid or semi-liquid sugar alcohol syrups in an amount of at most 7% by weight of the core. In some embodiments, the core further comprises one or more liquid or semi-liquid sugar alcohol syrups in an amount of at most 6% by weight of the core. In some embodiments, the core further comprises one or more liquid or semi-liquid sugar alcohol syrups in an amount of at most 5% by weight of the core. In some embodiments, the core further comprises one or more liquid or semi-liquid sugar alcohol syrups in an amount of at most 4% by weight of the core. In some embodiments, the core further comprises one or more liquid or semi-liquid sugar alcohol syrups in an amount of at most 3% by weight of the core. In some embodiments, the core further comprises one or more liquid or semi-liquid sugar alcohol syrups in an amount of at most 2% by weight of the core.

In some embodiments, the core further comprises one or more liquid or semi-liquid sugar alcohol syrups or sugar syrups providing binding properties and improved coherency to the mass of the core.

In some embodiments, the core further comprises one or more liquid or semi-liquid sugar alcohol syrups comprising maltitol syrup, also known as hydrolyzed starch hydrolysate (HSH).

In some embodiments, the core further comprises one or more liquid or semi-liquid sugar syrups comprising glucose.

In some embodiments, the core further comprises one or more liquid or semi-liquid sugar syrups providing stickiness to the mass of the core. In some embodiments of the invention, stickiness is not preferred. In some other embodiments, stickiness is acceptable.

In some embodiments, the core further comprises cocoa. In some embodiments, the core further comprises cocoa in an amount of 1 to 7% by weight of the core. In some embodiments, the core further comprises cocoa. In some embodiments, the core further comprises cocoa in an amount of 1 to 5% by weight of the core. In some embodiments, the core further comprises cocoa in an amount of 2 to 5% by weight of the core.

In some embodiments, the one or more powdered proteins is present in an amount of 10 to 55% by weight of the core.

In some embodiments, the one or more powdered proteins is present in an amount of 10 to 50% by weight of the core.

In some embodiments, the one or more powdered proteins is present in an amount of 10 to 40% by weight of the core.

In some embodiments, the one or more powdered proteins is present in an amount of 20 to 30% by weight of the core.

In some embodiments, the one or more powdered proteins is present in an amount of 20 to 45% by weight of the core.

In some embodiments, the one or more powdered proteins is present in an amount of 30 to 45% by weight of the core.

In some embodiments, the one or more powdered proteins is substantially taste neutral.

In some embodiments, the one or more powdered proteins partly replaces powdered sugars or powdered sugar alcohols.

In some embodiments, the one or more powdered proteins replaces powdered sugars or powdered sugar alcohols.

In some embodiments, the one or more powdered proteins comprises dairy proteins.

In some embodiments, the one or more powdered proteins comprises a mixture of dairy proteins.

In some embodiments, the one or more powdered proteins comprises isolated, concentrated or hydrolyzed dairy proteins.

In some embodiments, the one or more powdered proteins comprises proteins selected from the group consisting of proteins originating from casein, calcium caseinate, whey, and combinations thereof.

In some embodiments, the one or more powdered proteins comprises plant proteins.

In some embodiments, the one or more powdered proteins comprises a mixture of plant proteins.

In some embodiments, the one or more powdered proteins comprises isolated, concentrated or hydrolyzed plant proteins.

In some embodiments, the one or more powdered proteins comprises plant proteins selected from the group consisting of proteins originating from pea, soy, faba beans, chia, rice, oat, wheat, potato, canola, sunflower, rapeseed, and combinations thereof.

In some embodiments, the one or more powdered proteins comprises a mixture of plant proteins and dairy proteins.

In some embodiments, the core further comprises one or more powdered sugar alcohols. In some embodiments, the core further comprises one or more powdered sugar alcohols present in an amount of 5 to 45% by weight of the core. In some embodiments, the core further comprises one or more powdered sugar alcohols. In some embodiments, the core further comprises one or more powdered sugar alcohols present in an amount of 5 to 40% by weight of the core. In some embodiments, the core further comprises one or more powdered sugar alcohols present in an amount of 5 to 30% by weight of the core. In some embodiments, the core further comprises one or more powdered sugar alcohols present in an amount of 10 to 40% by weight of the core. In some embodiments, the core further comprises one or more powdered sugar alcohols present in an amount of 10 to 30% by weight of the core.

In some embodiments, the core further comprises one or more powdered sugar alcohols present in an amount of 1 to 25% by weight of the core. In some embodiments, the core further comprises one or more powdered sugar alcohols present in an amount of 2 to 20% by weight of the core. In some embodiments, the core further comprises one or more powdered sugar alcohols present in an amount of 3 to 15% by weight of the core. In some embodiments, the core further comprises one or more powdered sugar alcohols present in an amount of 3 to 10% by weight of the core.

In some embodiments, the core further comprises one or more powdered sugar alcohols selected from the group consisting of xylitol, maltitol, mannitol, erythritol, isomalt, sorbitol, lactitol, and any combination thereof.

In some embodiments, the core further comprises one or more powdered sugar alcohols selected from the group consisting of maltitol, isomalt, sorbitol, and any combination thereof.

In some embodiments, the one or more powdered sugar alcohols comprises maltitol. In some embodiments, the one or more powdered sugar alcohols is maltitol.

In some embodiments, the one or more powdered sugar alcohols is maltitol and wherein the one or more indigestible water soluble fibers is indigestible maltodextrin.

In some embodiments, the one or more powdered sugar alcohols comprises sorbitol. In some embodiments, the one or more powdered sugar alcohols is sorbitol.

In some embodiments, the one or more powdered sugar alcohols comprises xylitol. In some embodiments, the one or more powdered sugar alcohols is xylitol.

In some embodiments, the one or more powdered sugar alcohols comprises isomalt. In some embodiments, the one or more powdered sugar alcohols is isomalt.

In some embodiments, the one or more powdered sugar alcohols comprises isomalt and the one or more indigestible water soluble fibers comprises inulin.

In some embodiments, the one or more powdered sugar alcohols comprises isomalt and the one or more indigestible water soluble fibers comprises indigestible maltodextrin.

In some embodiments, the one or more powdered sugar alcohols is micro-pulverized. According to the invention, micro-pulverization may provide a further improvement of the coherency of the soft chew tablets according to the invention. Micro-crystallization may also establish a greater similarity to traditional soft chew tablets that have been produced by cooking. This was highly unexpected by the inventors.

In some embodiments, the one or more powdered sugar alcohols has an average particle size of less than 100 microns, such as less than 75 microns, such as less than 50 microns. The average particles size being measured by standard laser diffraction techniques.

In some embodiments, the one or more powdered sugar alcohols passes a screen cloth having a mesh opening of 0.074 mm (ASTM 200 mesh).

In some embodiments, the one or more indigestible water soluble fibers and the one or more powdered sugar alcohols are present in a range of 1:3 to 10:1. In some embodiments, the one or more indigestible water soluble fibers and the one or more powdered sugar alcohols are present in a range of 1:2 to 10:1. In some embodiments, the one or more indigestible water soluble fibers and the one or more powdered sugar alcohols are present in a range of 1:1 to 10:1.

In some embodiments, the core further comprises inorganic filler.

In some embodiments, the core further comprises one or more inorganic mineral fillers with a BET specific surface area above 15 m2/g, wherein the BET specific surface area is measured in accordance with ISO 9277, and wherein the one or more inorganic mineral fillers comprises calcium carbonate.

In some embodiments, the core further comprises one or more inorganic mineral fillers with a BET specific surface area above 15 m2/g, wherein the BET specific surface area is measured in accordance with ISO 9277, and wherein the one or more inorganic mineral fillers comprises calcium carbonate in an amount of 70-90% by weight of the inorganic mineral filler.

In some embodiments, the core further comprises one or more inorganic mineral fillers with a BET specific surface area above 15 m2/g, wherein the BET specific surface area is measured in accordance with ISO 9277, and wherein the one or more inorganic mineral fillers comprises calcium carbonate in an amount of 40-60% by weight of the inorganic mineral filler.

Addition of one or more inorganic mineral fillers with a BET specific surface area above 15 m2/g is particularly advantageous when applying relative high dose actives in a liquid state, such as omega fatty acid oils and vegetable oils.

In some embodiments, the core further comprises talc. In some embodiments, the core further comprises calcium carbonate. In some embodiments, the core further comprises calcium carbonate in an amount of 5-20% by weight of the core. In some embodiments, the core further comprises calcium carbonate in an amount of 5-25% by weight of the core. In some embodiments, the core further comprises calcium carbonate in an amount of 5-30% by weight of the core. In some embodiments, the core further comprises calcium carbonate in an amount of 5-35% by weight of the core. In some embodiments, the core further comprises calcium carbonate in an amount of 10-20% by weight of the core. Addition of calcium carbonate is beneficial in order to provide an improved texture of the product.

In some embodiments, the core comprises water in an amount of no more than 8% by weight of the core before formation of the core, such as no more than 6% by weight. In some embodiments, the core comprises water in an amount of no more than 10% by weight of the core before formation of the core. In some embodiments, the core comprises water in an amount of no more than 6% by weight of the core before formation of the core.

In some embodiments, the core comprises unbound water in an amount of no more than 10% by weight of the core before formation of the core. In some embodiments, the core comprises unbound water in an amount of no more than 8% by weight of the core before formation of the core. In some embodiments, the core comprises unbound water in an amount of no more than 6% by weight of the core before formation of the core, such as no more than 4% by weight, such as no more than 2% by weight.

In some embodiments, the core comprises water that provides binding properties to the core during formation.

In some embodiments, the core has a water activity of less than 0.6, such as less than 0.55, such as less than 0.5.

In some embodiments, the one or more texturizers is present in an amount of 0.1 to 22% by weight of the core. In some embodiments, the one or more texturizers is present in an amount of 0.1 to 20% by weight of the core. In some embodiments, the one or more texturizers is present in an amount of 0.1 to 18% by weight of the core. In some embodiments, the one or more texturizers is present in an amount of 0.5 to 25% by weight of the core. In some embodiments, the one or more texturizers is present in an amount of 0.5 to 22% by weight of the core. In some embodiments, the one or more texturizers is present in an amount of 0.5 to 20% by weight of the core. In some embodiments, the one or more texturizers is present in an amount of 0.5 to 18% by weight of the core. In some embodiments, the one or more texturizers is present in an amount of 0.5 to 15% by weight of the core. In some embodiments, the one or more texturizers is present in an amount of 1 to 12% by weight of the core. In some embodiments, the one or more texturizers is present in an amount of 1 to 15% by weight of the core. In some embodiments, the one or more texturizers is present in an amount of 5 to 15% by weight of the core. In some embodiments, the one or more texturizers is present in an amount of 10 to 15% by weight of the core. In some embodiments, the one or more texturizers is present in an amount of 2 to 10% by weight of the core. In some embodiments, the one or more texturizers is present in an amount of 1 to 10% by weight of the core. In some embodiments, the one or more texturizers is present in an amount of 0.5 to 8% by weight of the core. In some embodiments, the one or more texturizers is present in an amount of 1 to 5% by weight of the core.

According to the invention, the texturizers according to the invention may provide a further improvement of the coherency of the soft chew tablets according to the invention. The texturizers may also establish a greater similarity to traditional soft chew tablets that have been produced by cooking without the drawbacks of traditional soft chew tablets. This was highly unexpected by the inventors.

In some embodiments, the one or more texturizers comprises one or more vegetable oils, such as hydrogenated vegetable oils. In the present context, “vegetable oil” is similar to “vegetable fat”.

In some embodiments, the one or more texturizers comprises one or more vegetable oils having a melting point below 40-50 degree Celsius.

In some embodiments, the one or more texturizers comprises one or more vegetable oils selected from the group consisting of coconut oil, palm oil, palm kernel oil, hydrogenated coconut oil, hydrogenated palm oil, hydrogenated palm kernel oil, and any combination thereof.

In some embodiments, the one or more texturizers comprises one or more medium chain triglycerides (MCT). In some embodiments, the one or more texturizers comprises one or more medium chain triglycerides (MCT) comprising C6-C12 triglycerides. In some embodiments, the one or more texturizers comprises one or more medium chain triglycerides (MCT) comprising C8-C10 triglycerides. In some embodiments, the one or more texturizers comprises one or more medium chain triglycerides (MCT) comprising capric acid. In some embodiments, the one or more texturizers comprises one or more medium chain triglycerides (MCT) comprising caprylic acid. In some embodiments, the one or more texturizers comprises one or more medium chain triglycerides (MCT) characterized by being liquid at 25 degrees Celsius.

In some embodiments, the one or more texturizers comprises one or more vegetable oils in an amount of 1 to 10% by weight of the core. In some embodiments, the one or more texturizers comprises one or more vegetable oils in an amount of 1 to 8% by weight of the core. In some embodiments, the one or more texturizers comprises one or more vegetable oils in an amount of 1 to 5% by weight of the core.

In some embodiments, the one or more texturizers comprises one or more vegetable oils in an amount of 2 to 4% by weight of the core.

In some embodiments, the one or more texturizers comprises one or more of lecithin, gelatine, acacia, or glycerin. In some embodiments, the one or more texturizers comprises lecithin. In some embodiments, the one or more texturizers comprises gelatine. In some embodiments, the one or more texturizers comprises acacia. In some embodiments, the one or more texturizers comprises glycerin. In some embodiments, the one or more texturizers comprises carrageenan. In some embodiments, the one or more texturizers comprises instant starch. Instant starch is also known as “pregelatinized starch”.

In some embodiments, the one or more texturizers comprises glycerin in an amount of 1 to 15% by weight of the core. In some embodiments, the one or more texturizers comprises glycerin in an amount of 1 to 12% by weight of the core. In some embodiments, the one or more texturizers comprises glycerin in an amount of 1 to 10% by weight of the core. In some embodiments, the one or more texturizers comprises glycerin in an amount of 1 to 8% by weight of the core. In some embodiments, the one or more texturizers comprises glycerin in an amount of 1 to 5% by weight of the core. In some embodiments, the one or more texturizers comprises glycerin in an amount of 1 to 3% by weight of the core.

In some embodiments, the one or more texturizers comprises lecithin. In some embodiments, the one or more texturizers comprises gelatine. In some embodiments, the one or more texturizers comprises carrageenan. Gelatine with a higher bloom number is presently preferred. In some embodiments, the one or more texturizers comprises acacia. In some embodiments, the one or more texturizers comprises a vegetable oil, lecithin, gelatine, acacia, and glycerin. In some embodiments, the one or more texturizers comprises a vegetable oil, lecithin, carrageenan, acacia, and glycerin.

In some embodiments, the one or more texturizers comprises gelatine in an amount of no more than 5% by weight of the core. In some embodiments, the one or more texturizers comprises gelatine in an amount of no more than 10% by weight of the core.

In some embodiments, the core further comprises one or more active ingredients present in an amount of 1 to 60% by weight of the core. In some embodiments, the core further comprises one or more active ingredients present in an amount of 10 to 60% by weight of the core. In some embodiments, the core further comprises one or more active ingredients present in an amount of 20 to 50% by weight of the core. In some embodiments, the core further comprises one or more active ingredients present in an amount of 30 to 60% by weight of the core.

In some embodiments, the one or more active ingredients comprises one or more antacids.

In some embodiments, the one or more antacids is present in an amount of 20 to 60% by weight of the core. In some embodiments, the one or more antacids is present in an amount of 30 to 60% by weight of the core. In some embodiments, the one or more antacids is present in an amount of 40 to 60% by weight of the core. In some embodiments, the one or more antacids is present in an amount of 10 to 50% by weight of the core. In some embodiments, the one or more antacids is present in an amount of 20 to 50% by weight of the core. In some embodiments, the one or more antacids is present in an amount of 30 to 50% by weight of the core. In some embodiments, the one or more antacids is present in an amount of 25 to 45% by weight of the core.

In some embodiments, the one or more antacids is present in an amount of 1 to 50% by weight of the core. In some embodiments, the one or more antacids is present in an amount of 1 to 40% by weight of the core. In some embodiments, the one or more antacids is present in an amount of 1 to 30% by weight of the core. In some embodiments, the one or more antacids is present in an amount of 1 to 25% by weight of the core. In some embodiments, the one or more antacids is present in an amount of 1 to 20% by weight of the core. In some embodiments, the one or more antacids is present in an amount of 1 to 15% by weight of the core. In some embodiments, the one or more antacids is present in an amount of 1 to 5% by weight of the core. In some embodiments, the one or more antacids is present in an amount of 2 to 5% by weight of the core.

In some embodiments, the one or more antacids is selected from the group consisting of calcium carbonate, magnesium carbonate, sodium bicarbonate, magnesium hydroxide, aluminum hydroxide, magnesium silicate, simethicone, bismuth subsalicylate, and combinations thereof. Sodium alginate may be added in addition to calcium carbonate as well as other ingredients and actives.

In some embodiments, the one or more antacids comprises calcium carbonate. In some embodiments, the one or more antacids consists of calcium carbonate. In some embodiments, the one or more antacids comprises calcium carbonate in an amount of 20 to 60% by weight of the core. In some embodiments, the one or more antacids comprises calcium carbonate in an amount of 20 to 50% by weight of the core. In some embodiments, the one or more antacids comprises calcium carbonate in an amount of 30 to 60% by weight of the core. In some embodiments, the one or more antacids comprises calcium carbonate in an amount of 40 to 60% by weight of the core.

In some embodiments, the core further comprises one or more active ingredients comprises one or more active ingredients with nutraceutical and/or pharmaceutical properties. In the present context, nutraceuticals is also known as dietary supplements.

In some embodiments, the one or more active ingredients comprises one or more lipophilic active ingredients. Lipophilic active ingredients comprise saturated, mono-unsaturated and polyunsaturated fatty acids.

In some embodiments, the one or more active ingredients comprises one or more electrolytic active ingredients. Electrolytic active ingredients include water soluble nutritive salts and may sometimes also be referred to as sports nutrition active ingredients.

In some embodiments, the one or more active ingredients comprises one or more energy stimulating active ingredients.

In some embodiments, the one or more active ingredients comprises one or more vitamins.

In some embodiments, the one or more active ingredients comprises one or more minerals.

In some embodiments, the one or more active ingredients comprises caffeine.

In some embodiments, the one or more active ingredients comprises L-theanine.

In some embodiments, the one or more active ingredients comprises collagen. In some embodiments, the one or more active ingredients comprises creatine. In some embodiments, the one or more active ingredients comprises BCAA. In some embodiments, the one or more active ingredients comprises milk proteins. In some embodiments, the one or more active ingredients comprises magnesium. In some embodiments, the one or more active ingredients comprises collagen. In some embodiments, the one or more active ingredients comprises hyaluronic acid. In some embodiments, the one or more active ingredients comprises keratin. In some embodiments, the one or more active ingredients comprises omega 3 fatty acids. In some embodiments, the one or more active ingredients comprises taurine.

In some embodiments, the one or more active ingredients does not comprise nicotine.

In some embodiments, the one or more active ingredients is present in an amount of 1 to 30% by weight of the core, such as 1 to 10% by weight of the core, such as 1 to 5% by weight of the core.

In some embodiments, the soft chew tablet disintegrates within 2 minutes upon oral administration.

In some embodiments, the soft chew tablet disintegrates within 1.5 minute upon oral administration.

In some embodiments, the soft chew tablet disintegrates within 1 minute upon oral administration.

In some embodiments, the soft chew tablet disintegrates within 30 seconds upon oral administration.

One of the advantages of the present invention is that disintegration of the soft chew tablet was seen to be faster than traditional soft chew tablets that have undergone a cooking process. In the present context, less than 2 minutes is considered to be fast disintegration, and particularly less than 1 minute is seen to be very fast disintegrating. This was highly unexpected and surprising to the inventors. Disintegration was expected to be the same level as traditional soft chew tablets where the ingredients have been heated at relative high temperatures.

In some embodiments, the core has a hardness of more than 17 N (Newton) as measured on a standard texture analyzer known to one skilled in the art. A hardness of less than 17 N would not be preferable according to the invention. A certain hardness is typically required to allow for proper coating of the core of the present invention. Typically, the inventors by measuring the hardness of the soft chew tablets were able to distinguish inferior cores with cores fulfilling the requirement of the present invention in terms of texture.

In some embodiments, the soft chew tablet does not comprise an outer coating. In some embodiments, the soft chew tablet comprises an outer coating. In some embodiments, the soft chew tablet comprises an outer coating in an amount of 1 to 40% by weight of the soft chew tablet.

In some embodiments, the outer coating, such as outer hard coating or chocolate coating, constitutes 2 to 40% by weight of the soft chew tablet. In some embodiments, the outer coating, such as outer hard coating or chocolate coating, constitutes 3 to 40% by weight of the soft chew tablet. the outer coating constitutes 5 to 40% by weight of the soft chew tablet. In some embodiments, the outer coating, such as outer hard coating or chocolate coating, constitutes 10 to 35% by weight of the soft chew tablet. In some embodiments, the outer coating, such as outer hard coating or chocolate coating, constitutes 15 to 30% by weight of the soft chew tablet. In some embodiments, the outer coating, such as outer hard coating or chocolate coating, constitutes 20 to 35% by weight of the soft chew tablet. In some embodiments, the outer coating, such as outer hard coating or chocolate coating, constitutes 20 to 30% by weight of the soft chew tablet.

In some embodiments, the soft chew tablet comprises an outer coating comprising a chocolate coating. In some embodiments, the soft chew tablet comprises an outer chocolate coating.

In some embodiments, the outer coating comprises a suspension of sugar applied to the core in a pan coating process. In some embodiments, the outer coating comprises a suspension of sugar alcohols applied to the core in a pan coating process, which is presently preferred.

In some embodiments, the outer coating comprises a suspension of sugar applied during multiple cycles to the core in a pan coating process, such as during 3 to 80 cycles, to provide a hard coating comprising multiples layers. In some embodiments, the outer coating comprises a suspension of sugar alcohols applied during multiple cycles to the core in a pan coating process, such as during 3 to 80 cycles, to provide a hard coating comprising multiples layers.

In the present context, the term “hard coating” is used in the conventional meaning of that term. The hard coating provides a crunchy layer, which is appreciated by the consumer and protects the cores of the soft chew tablets as seen according to the invention. In a typical process of providing the cores with a protective sugar alcohol coating, the cores are successively treated in suitable coating equipment with aqueous solutions of crystallizable sugar alcohols, such as maltitol or xylitol, which, depending on the stage of coating reached, may contain other functional ingredients, e.g., fillers, binding agents, flavors, colors, etc. In the present context, “functional ingredients” is not intended to cover active ingredients, such as “nutraceutical ingredients” or the like.

In a typical hard coating process as it will be described in detail in the following, a suspension containing crystallizable sugar and/or polyol is applied onto the cores and the water it contains is evaporated off by blowing with air. This cycle must be repeated several times, typically 3 to 80 times, in order to reach the swelling required. The term “swelling” refers to the increase in weight or thickness of the products, as considered at the end of the coating operation by comparison with the beginning, and in relation to the final weight or thickness of the coated products.

In some embodiments, the outer coating comprises one or more fillers, binding agents, colors, or flavors.

In some embodiments, the outer coating comprises binding agents, such as acacia or gelatine. In some embodiments, the outer hard coating comprises binding agents including acacia. In some embodiments, the outer hard coating comprises binding agents including gelatine.

In some embodiments, the outer coating comprises binding agents in an amount of 1 to 10% by weight of the coating. In some embodiments, the outer coating comprises binding agents in an amount of 2 to 10% by weight of the coating. In some embodiments, the outer coating comprises binding agents in an amount of 2 to 8% by weight of the coating. In some embodiments, the outer coating comprises binding agents in an amount of 3 to 8% by weight of the coating. In some embodiments, the outer coating comprises binding agents in an amount of 4 to 7% by weight of the coating. The amount of binding agents are based on dry weight.

In some embodiments, the outer coating comprises sugar alcohol selected from the group consisting of isomalt, maltitol, sorbitol, xylitol, and any combination thereof. In some embodiments, the outer coating comprises isomalt. In some embodiments, the outer coating comprises maltitol. In some embodiments, the outer coating comprises sorbitol. In some embodiments, the outer coating comprises xylitol.

In some embodiments, the outer coating comprises sugar alcohol in an amount of 30 to 98% by weight of the coating, such as in an amount of 50 to 95% by weight of the coating. In some embodiments, the outer coating comprises sugar alcohol in an amount of 30 to 95% by weight of the coating, such as in an amount of 50 to 95% by weight of the coating. The percentages are based on dry weight.

In some embodiments, the outer coating comprises sugar alcohol in an amount of 30 to 98% by weight of the coating. In some embodiments, the outer coating comprises sugar alcohol in an amount of 40 to 98% by weight of the coating. In some embodiments, the outer coating comprises sugar alcohol in an amount of 50 to 98% by weight of the coating. In some embodiments, the outer coating comprises sugar alcohol in an amount of 50 to 95% by weight of the coating. In some embodiments, the outer coating comprises sugar alcohol in an amount of 60 to 95% by weight of the coating. In some embodiments, the outer coating comprises sugar alcohol in an amount of 70 to 95% by weight of the coating. In some embodiments, the outer coating comprises sugar alcohol in an amount of 80 to 95% by weight of the coating. In some embodiments, the outer coating comprises sugar alcohol in an amount of 90 to 95% by weight of the coating.

In some embodiments, the outer coating, such as hard coating or chocolate coating, constitutes a partial moisture barrier to ingredients in the core of the soft chew tablet. In the present context, “partial moisture barrier” is intended to mean that the coating, such as hard coating or chocolate coating, does not necessarily invoke 100% barrier properties, while this is preferred, but may invoke less than 100% barrier properties. In some embodiments, “partial” implies more than 100% barrier properties calculated over 7 days of storage, excluding any moisture absorbed from the coating during processing. In some embodiments, “partial” implies more than 99% barrier properties. In some embodiments, “partial” implies more than 98% barrier properties. In some embodiments, “partial” implies more than 95% barrier properties. In some embodiments, “partial” implies more than 90% barrier properties. In some embodiments, “partial” implies more than 80% barrier properties. It is implied that “partial moisture barrier” may also be understood as “improvement of the moisture retention in the core”.

In some embodiments, the outer coating, such as hard coating or chocolate coating, constitutes an anti-sticking barrier to ingredients in the core of the soft chew tablet. One of the challenges of the prior art is that the core may be sticky, which causes challenges in terms of handling of the products. It was a surprise to the inventors of the invention that the problems with stickiness could be solved by providing a coating, such as hard coating or chocolate coating, to the cores according to the invention. Due to the relative wet conditions in the coating process, such as hard coating or chocolate coating, it was expected that the core would be compromised by the coating process, including the texture. This was particularly expected for a hard coating and chocolate coating. However, this was not seen. Hence, the coating process of the invention, such as hard coating or chocolate coating, may be seen to solve several challenges, including the moisture balance as well as challenges with stickiness.

In some embodiments, the outer coating is a hard coating. In some embodiments, the outer coating is a coating selected from the group consisting of a film coating, a soft coating, and a chocolate coating.

In some embodiments, the soft chew tablet further comprises a sub-coat applied between the core and the outer coating, such as hard coating.

In some embodiments, the soft chew tablet further comprises a sub-coat applied between the core and the outer hard coating constituting 0.1 to 5% by weight of the soft chew tablet. In some embodiments, the soft chew tablet further comprises a sub-coat applied between the core and the outer hard coating constituting 0.1 to 4% by weight of the soft chew tablet. In some embodiments, the soft chew tablet further comprises a sub-coat applied between the core and the outer hard coating constituting 0.2 to 4% by weight of the soft chew tablet. In some embodiments, the soft chew tablet further comprises a sub-coat applied between the core and the outer hard coating constituting 0.2 to 3% by weight of the soft chew tablet. In some embodiments, the soft chew tablet further comprises a sub-coat applied between the core and the outer hard coating constituting 0.3 to 3% by weight of the soft chew tablet. In some embodiments, the soft chew tablet further comprises a sub-coat applied between the core and the outer hard coating constituting 0.5 to 3% by weight of the soft chew tablet. In some embodiments, the soft chew tablet further comprises a sub-coat applied between the core and the outer hard coating constituting 1 to 3% by weight of the soft chew tablet.

In some embodiments, the soft chew tablet further comprises a sub-coat applied between the core and the outer hard coating comprising indigestible maltodextrin. In some embodiments, the soft chew tablet further comprises a sub-coat applied between the core and the outer hard coating comprising acacia. In some embodiments, the soft chew tablet further comprises a sub-coat applied between the core and the outer hard coating comprising maltitol. In some embodiments, the soft chew tablet further comprises a sub-coat applied between the core and the outer hard coating comprising shellac. Typically, the sub-coat (also known as pre-coat) is applied in a similar way as a film coating. In some embodiments, the sub-coat is a “film coating”. In some embodiments, another “film coating” may be applied on top of this film-coating to provided dual film coating, or multiple film coatings.

In further useful embodiments, the core of the soft chew tablets according to the invention is an element that is subjected to a film coating process and which therefore comprises one or more film-forming polymeric agents and optionally one or more auxiliary compounds, e.g., plasticizers, pigments and opacifiers. A film coating is a thin polymer-based coating applied to a core of any of the above forms. The thickness of such a coating is usually between 20 and 100 km. Generally, the film coating is obtained by passing the cores through a spray zone with atomized droplets of the coating materials in a suitable aqueous or organic solvent vehicle, after which the material adhering to the cores is dried before the next portion of coating is received. This cycle is repeated until the coating is complete.

In the present context, suitable film-coating polymers include edible cellulose derivatives such as cellulose ethers including methylcellulose (MC), hydroxyethyl cellulose (HEC), hydroxypropyl cellulose (HPC) and hydroxypropyl methylcellulose (HPMC). Other useful film-coating agents are acrylic polymers and copolymers, e.g. methylacrylate aminoester copolymer or mixtures of cellulose derivatives and acrylic polymers. A particular group of film-coating polymers, also referred to as functional polymers are polymers that, in addition to its film-forming characteristics, confer a modified release performance with respect to active components of the cores. Such release modifying polymers include methylacrylate ester copolymers, ethylcellulose (EC) and enteric polymers designed to resist the acidic stomach environment. The latter group of polymers include: cellulose acetate phtalate (CAP), polyvinyl acetate phtalate (PVAP), shellac, metacrylic acid copolymers, cellulose acetate trimellitate (CAT) and HPMC. It will be appreciated that the outer film coating according to the present invention may comprise any combination of the above film-coating polymers as well as the components also mentioned elsewhere.

In some embodiments, the soft chew tablet further comprises a sub-coat applied between the core and the outer hard coating constituting a partial moisture barrier to ingredients in the core of the soft chew tablet.

In the present context, “partial moisture barrier” is intended to mean that the sub-coat does not necessarily invoke 100% barrier properties, while this is preferred, but may invoke less than 100% barrier properties. In some embodiments, “partial” implies more than 100% barrier properties calculated over 7 days of storage, excluding any moisture absorbed from the coating during processing. In some embodiments, “partial” implies more than 99% barrier properties. In some embodiments, “partial” implies more than 98% barrier properties. In some embodiments, “partial” implies more than 95% barrier properties. In some embodiments, “partial” implies more than 90% barrier properties. In some embodiments, “partial” implies more than 80% barrier properties. It is implied that “partial moisture barrier” may also be understood as “improvement of the moisture retention in the core”.

In some embodiments, the sub-coat constitutes an anti-sticking barrier to ingredients in the core of the soft chew tablet. One of the challenges of the prior art is that the core may be sticky, which causes challenges in terms of handling of the products. It was a surprise to the inventors of the invention that the problems with stickiness could be solved by providing a sub-coat to the cores according to the invention. Due to the relative wet conditions in the coating process, it was expected that the core would be compromised by the coating process, including the texture. However, this was not seen. Hence, the hard coating process of the invention may be seen to solve several challenges, including the moisture balance as well as challenges with stickiness.

In some embodiments, the soft chew tablet comprises the core in an amount of 60 to 100% by weight of the soft chew tablet. In some embodiments, the soft chew tablet comprises the core in an amount of 60 to 99% by weight of the soft chew tablet.

In some embodiments, the soft chew tablet comprises the core in an amount of 60 to 99% by weight of the soft chew tablet. In some embodiments, the soft chew tablet comprises the core in an amount of 60 to 98% by weight of the soft chew tablet. In some embodiments, the soft chew tablet comprises the core in an amount of 60 to 95% by weight of the soft chew tablet. In some embodiments, the soft chew tablet comprises the core in an amount of 60 to 90% by weight of the soft chew tablet. In some embodiments, the soft chew tablet comprises the core in an amount of 70 to 95% by weight of the soft chew tablet. In some embodiments, the soft chew tablet comprises the core in an amount of 70 to 90% by weight of the soft chew tablet. In some embodiments, the soft chew tablet comprises the core in an amount of 60 to 90% by weight of the soft chew tablet. In some embodiments, the soft chew tablet comprises the core in an amount of 60 to 80% by weight of the soft chew tablet.

### DETAILED DESCRIPTION

The invention will now be described in more details with respect to certain aspects and embodiments of the invention. These aspects and embodiments are intended to be understood in connection with the rest of the description, including the Summary of the Invention and the Examples of the invention.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein, the term “%” and “percent” refers to percent by weight, unless otherwise is stated.

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”. Additionally, the words “a” and “an” when used in the present document in connection with the word comprising or containing denote “one or more.” The expression “one or more” is intended to mean one, two, three or more.

The term “particle size” relates to the ability of the particles to move through or be retained by sieve holes of a specific size unless otherwise stated. As used herein, the term “particle size” refers to the average particle size as determined according to European Pharmacopoeia 9.1 when using test method 2.9.38 particle size distribution estimation by analytical sieving, unless otherwise specifically is mentioned.

The term “particle” or similar wording is intended to denote a single, discrete composition of solid matter, such as a granule or individual elements in powder, having a certain size that may deviate considerable.

By the phrase “texture” is meant a qualitative measure of the properties of the oral tablet and of the overall mouth-feel experienced by the user during use. Thus, the term “texture” encompasses measurable quantities such as hardness as well as more subjective parameters related to the feel experienced by a user.

The term “release” in the present context is intended to mean under “in vitro” conditions if not stated otherwise. In particular, the “release rate” during a certain period of time is intended to mean the amount in percentage of active ingredients that is released during the period. In the present context the term “release” refers to the released substance being liberated from the water-soluble matrix. In some embodiments, the process of releasing a substance corresponds to the substance being dissolved in saliva.

As used herein, the term “disintegrate” refers to a reduction of an object to components, fragments or particles. Disintegration time may be measured in vitro or in vivo. Unless otherwise stated, the in vitro measurements are carried out in accordance to European Pharmacopeia 9.0, section 2.9.1, Disintegration of tablets and capsules.

As used herein, the term “dissolve” is the process where a solid substance enters a solvent (oral saliva) to yield a solution. Unless otherwise stated, dissolving implies a full dissolving of the compound in question.

A “self-emulsifying agent” (as contained in SEDDS) is an agent which will form an emulsion when presented with an alternate phase with a minimum energy requirement. In contrast, an emulsifying agent, as opposed to a self-emulsifying agent, is one requiring additional energy to form an emulsion.

When referring to amounts of an ingredient by terms such as “less than”, “no more than”, this generally refers to the particular ingredient being absent or present in a range from trace amounts to the specified maximum amount.

As used herein the term “flavor” is understood as having its ordinary meaning within the art. Flavor includes liquid and powdered flavors. Thus, flavors do of course not include sweeteners (such as sugar, sugar alcohols and high intensity sweeteners), or acids providing pure acidity/sourness, nor compounds providing pure saltiness (e.g. NaCl) or pure bitterness. The flavors can be natural or synthetic flavors.

High intensity artificial sweetening agents may be applied according to the invention. For example, high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 8% by weight (such as from about 0.02 to about 8% by weight).

Usable flavors including as examples almond, almond amaretto, apple, Bavarian cream, black cherry, black sesame seed, blueberry, brown sugar, bubblegum, butterscotch, cappuccino, caramel, caramel cappuccino, cheesecake (graham crust), cinnamon redhots, cotton candy, circus cotton candy, clove, coconut, coffee, clear coffee, double chocolate, energy cow, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry, kiwi, tropical punch, tutti frutti, vanilla, or any combination thereof.

In one embodiment the tablet according to the invention comprises a pharmaceutically, cosmetically or biologically active substance. Examples of such active substances, a comprehensive list of which is found e.g. in WO 00/25598, which is incorporated herein by reference, include drugs, dietary supplements, antiseptic agents, pH adjusting agents, anti-smoking agents and substances for the care or treatment of the oral cavity and the teeth such as hydrogen peroxide and compounds capable of releasing urea during chewing. Examples of useful active substances in the form of antiseptics include salts and derivatives of guanidine and biguanidine (for instance chlorhexidine diacetate) and the following types of substances with limited water-solubility: quaternary ammonium compounds (e.g. ceramine, chloroxylenol, crystal violet, chloramine), aldehydes (e.g. paraformaldehyde), derivatives of dequaline, polynoxyline, phenols (e.g. thymol, p-chlorophenol, cresol), hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. also Martindale, The Extra Pharmacopoeia, 28th edition, pages 547-578; metal salts, complexes and compounds with limited water-solubility, such as aluminum salts, (for instance aluminum potassium sulphate AlK(SO4)2, 12H2O) and salts, complexes and compounds of boron, barium, strontium, iron, calcium, zinc, (zinc acetate, zinc chloride, zinc gluconate), copper (copper chloride, copper sulphate), lead, silver, magnesium, sodium, potassium, lithium, molybdenum, vanadium should be included; other compositions for the care of mouth and teeth: for instance; salts, complexes and compounds containing fluorine (such as sodium fluoride, sodium monofluorophosphate, aminofluorides, stannous fluoride), phosphates, carbonates and selenium. Further active substances can be found in J. Dent. Res. Vol. 28 No. 2, pages 160-171, 1949.

Examples of active substances in the form of agents adjusting the pH in the oral cavity include: acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulphates or oxides of sodium, potassium, ammonium, magnesium or calcium, especially magnesium and calcium.

Active ingredients may comprise the below mentioned compounds or derivates thereof but are not limited thereto: Acetaminophen, Acetylsalicylic acid, Buprenorphine, Bromhexin, Celcoxib, Codeine, Diphenhydramin, Diclofenac, Etoricoxib, Ibuprofen, Indometacin, Ketoprofen, Lumiracoxib, Morphine, Naproxen, Oxycodon, Parecoxib, Piroxicam, Pseudoefedrin, Rofecoxib, Tenoxicam, Tramadol, Valdecoxib, Calciumcarbonat, Magaldrate, Disulfiram, Bupropion, Nicotine, Azithromycin, Clarithromycin, Clotrimazole, Erythromycin, Tetracycline, Granisetron, Ondansetrone, Prometazin, Tropisetron, Brompheniramine, Ceterizin, leco-Ceterizin, Chlorcyclizine, Chlorpheniramin, Chlorpheniramin, Difenhydramine, Doxylamine, Fenofenadin, Guaifenesin, Loratidin, des-Loratidin, Phenyltoloxamine, Promethazin, Pyridamine, Terfenadin, Troxerutin, Methyldopa, Methylphenidate, Benzalcon. Chloride, Benzeth. Chloride, Cetylpyrid. Chloride, Chlorhexidine, Ecabet-sodium, Haloperidol, Allopurinol, Colchinine, Theophylline, Propanolol, Prednisolone, Prednisone, Fluoride, Urea, Actot, Glibenclamide, Glipizide, Metformin, Miglitol, Repaglinide, Rosiglitazone, Apomorfin, Cialis, Sildenafil, Vardenafil, Diphenoxylate, Simethicone, Cimetidine, Famotidine, Ranitidine, Ratinidine, cetrizin, Loratadine, Aspirin, Benzocaine, Dextrometorphan, Phenylpropanolamine, Pseudoephedrine, Cisapride, Domperidone, Metoclopramide, Acyclovir, Dioctylsulfosucc, Phenolphtalein, Almotriptan, Eletriptan, Ergotamine, Migea, Naratriptan, Rizatriptan, Sumatriptan, Zolmitriptan, Aluminum salts, Calcium salts, Ferro salts, Ag-salts, Zinc-salts, Amphotericin B, Chlorhexidine, Miconazole, Triamcinolonacetonid, Melatonine, Phenobarbitol, Caffeine, Benzodiazepiner, Hydroxyzine, Meprobamate, Phenothiazine, Buclizine, Brometazine, Cinnarizine, Cyclizine, Difenhydramine, Dimenhydrinate, Buflomedil, Amphetamine, Caffeine, Ephedrine, Orlistat, Phenylephedrine, Phenylpropanolamin, Pseudoephedrine, Sibutramin, Ketoconazole, Nitroglycerin, Nystatin, Progesterone, Testosterone, Vitamin B12, Vitamin C, Vitamin A, Vitamin D, Vitamin E, Pilocarpin, Aluminumaminoacetat, Cimetidine, Esomeprazole, Famotidine, Lansoprazole, Magnesiumoxide, Nizatide and or Ratinidine.

The invention is suitable for increased or accelerated release of active agents selected among the group of dietary supplements, oral and dental compositions, antiseptic agents, pH adjusting agents, anti-smoking agents, sweeteners, flavorings, aroma agents or drugs. Some of those will be described below.

The active agents to be used in connection with the present invention may be any substance desired to be released from the tablet. The active agents, for which a controlled and/or accelerated rate of release is desired, are primarily substances with a limited water-solubility, typically below 10 g/100 mL inclusive of substances which are totally water-insoluble. Examples are medicines, dietary supplements, oral compositions, anti-smoking agents, highly potent sweeteners, pH adjusting agents, flavorings etc.

Other active ingredients are, for instance, paracetamol, benzocaine, cinnarizine, menthol, carvone, caffeine, chlorhexidine-di-acetate, cyclizine hydrochloride, 1,8-cineol, nandrolone, miconazole, mystatine, sodium fluoride, nicotine, cetylpyridinium chloride, other quaternary ammonium compounds, vitamin E, vitamin A, vitamin D, glibenclamide or derivatives thereof, progesterone, acetylsalicylic acid, dimenhydrinate, cyclizine, metronidazole, sodium hydrogen carbonate, the active components from ginkgo, the active components from propolis, the active components from ginseng, methadone, oil of peppermint, salicylamide, hydrocortisone or astemizole.

Examples of active agents in the form of dietary supplements are for instance salts and compounds having the nutritive effect of vitamin B2 (riboflavin), B12, folinic acid, folic acid, niacine, biotine, poorly soluble glycerophosphates, amino acids, the vitamins A, D, E and K, minerals in the form of salts, complexes and compounds containing calcium, phosphorus, magnesium, iron, zinc, copper, iodine, manganese, chromium, selenium, molybdenum, potassium, sodium or cobalt.

Furthermore, reference is made to lists of nutritionists accepted by the authorities in different countries such as for instance US code of Federal Regulations, Title 21, Section 182.5013.182 5997 and 182.8013-182.8997.

Examples of active agents in the form of antiseptics are for instance salts and compounds of guanidine and biguanidine (for instance chlorhexidine diacetate) and the following types of substances with limited water-solubility: quaternary ammonium compounds (for instance ceramine, chloroxylenol, crystal violet, chloramine), aldehydes (for instance paraformaldehyde), compounds of dequaline, polynoxyline, phenols (for instance thymol, para chlorophenol, cresol) hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. furthermore Martindale, The Extra Pharmacopoeia, 28th edition, pages 547-578; metal salts, complexes and compounds with limited water-solubility, such as aluminum salts, (for instance aluminum potassium sulphate AlK(SO4)2,12H2O) and furthermore salts, complexes and compounds of boron, barium, strontium, iron, calcium, zinc, (zinc acetate, zinc chloride, zinc gluconate), copper (copper chloride, copper sulfate), lead, silver, magnesium, sodium, potassium, lithium, molybdenum, vanadium should be included; other compositions for the care of mouth and teeth: for instance; salts, complexes and compounds containing fluorine (such as sodium fluoride, sodiummonofluorophosphate, amino fluorides, stannous fluoride), phosphates, carbonates and selenium.

Cf. furthermore J. Dent. Res. Vol. 28 No. 2, pages 160-171, 1949, wherein a wide range of tested compounds is mentioned.

Examples of active agents in the form of agents adjusting the pH in the oral cavity include for instance: acceptable acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulfates or oxides of sodium, potassium, ammonium, magnesium or calcium, especially magnesium and calcium.

Examples of active agents in the form of anti-smoking agents include for instance: nicotine, tobacco powder or silver salts, for instance silver acetate, silver carbonate and silver nitrate.

Further examples of active agents are medicines of any type.

Examples of active agents in the form of medicines include caffeine, salicylic acid, salicyl amide and related substances (acetylsalicylic acid, choline salicylate, magnesium salicylate, sodium salicylate), paracetamol, salts of pentazocine (pentazocine hydrochloride and pentazocinelactate), buprenorphine hydrochloride, codeine hydrochloride and codeine phosphate, morphine and morphine salts (hydrochloride, sulfate, tartrate), methadone hydrochloride, ketobemidone and salts of ketobemidone (hydrochloride), beta-blockers, (propranolol), calcium antagonists, verapamil hydrochloride, nifedinpine as well as suitable substances and salts thereof mentioned in Pharm. Int., Nov. 85, pages 267-271, Barney H. Hunter and Robert L. Talbert, nitroglycerine, erythrityl tetranitrate, strychnine and salts thereof, lidocaine, tetracaine hydrochloride, etorphine hydrochloride, atropine, insulin, enzymes (for instance papain, trypsin, amyloglucosidase, glucoseoxidase, streptokinase, streptodornase, dextranase, alpha amylase), polypeptides (oxytocin, gonadorelin, (LH.RH), desmopressin acetate (DDAVP), isoxsuprine hydrochloride, ergotamine compounds, chloroquine (phosphate, sulfate), isosorbide, demoxytocin, heparin.

Other active ingredients include beta-lupeol, Letigen®, Sildenafil citrate and derivatives thereof.

Further examples of active ingredients include dental products including Carbamide, CPP Caseine Phospho Peptide; Chlorhexidine, Chlorhexidine di acetate, Chlorhexidine Chloride, Chlorhexidine di gluconate, Hexetedine, Strontium chloride, Potassium Chloride, Sodium bicarbonate, Sodium carbonate, Fluor containing ingredients, Fluorides, Sodium fluoride, Aluminum fluoride.

Further examples of active ingredients include Ammonium fluoride, Calcium fluoride, Stannous fluoride, Other fluor containing ingredients Ammonium fluorosilicate, Potassium fluorosilicate, Sodium fluorosilicate, Ammonium monofluorphosphate, Calcium monofluorphosphate, Potassium monofluorphosphate, Sodium monofluorphosphate, Octadecentyl Ammonium fluoride, Stearyl Trihydroxyethyl Propylenediamine Dihydrofluoride

Further examples of active ingredients include vitamins. Vitamins include A, B1, B2, B6, B12, Folinic acid, Folic acid, niacin, Pantothenic acid, biotine, C, D, E, K. Minerals include Calcium, phosphor, magnesium, iron, Zinc, Copper, Iod, Mangan, Crom, Selene, Molybden. Other active ingredients include: Q10®, enzymes. Natural drugs including Ginkgo Biloba, ginger, and fish oil.

Further examples of active ingredients include migraine drugs such as Serotonin antagonists: Sumatriptan, Zolmitriptan, Naratriptan, Rizatriptan, Eletriptan; nausea drugs such as Cyclizin, Cinnarizin, Dimenhydramin, Difenhydrinat; hay fever drugs such as Cetrizin, Loratidin, pain relief drugs such as Buprenorfin, Tramadol, oral disease drugs such as Miconazol, Amphotericin B, Triamcinolonaceton; and the drugs Cisaprid, Domperidon, Metoclopramid.

In an advantageous embodiment of the invention the active ingredient is selected from active ingredients for the throat selected from acetylcysteine, ambroxol, amylmetacresol, benzocaine, bisacodyl, bismuth subsalicylate, bromhexine, cetirizine, cetylpyridinium, chlorhexidine, dextromethorphan hydrobromide, 2,4-dichlorobenzyl alcohol, doxylamine succinate, eucalyptus oil, flurbiprofen, glycerin, hexylresorcinol, lidocaine, menthol, myrrh, paracetamol, pectin, peppermint oil, phenol, phenylephrine, povidone-iodine, pseudoephedrine, ranitidine, simethicone, sodium docusate, spearmint, zinc, or any combination thereof; active ingredients for the gastrointestinal tract selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol (acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, or any combination thereof, and active ingredients for buccal absorption selected from atenolol, baclofen, caffeine, carvedilol, chlorpheniramine, chlorpheniramine maleate, fluticasone propionate, maleate, desmopressin, diltiazem hydrochloride, doxylamine succinate, mycostatin, nicotine, nifedipine, nitroglycerin, omeprazole, ondansetron, oxymetazoline HCl, oxytocin, phenylephrine, piroxicam, prednisone, propranolol, salbutamol sulphate, scopoloamine butyl hydroxide, sumatriptan, triamcinolonacetonid, and any combination thereof.

The following non-limiting examples illustrate different variations of the present invention. The examples are meant for indicating the inventive concept; hence the mentioned examples should not be understood as exhaustive for the present.

### EXAMPLES

### Example 1

### Preparation of Soft Chew Cores

Compositions were prepared in a process of low speed shear mixing. The mixing process was conducted in a 2000 g Sigma blade kneader mixer operated with a speed of 40-50 rpm and a mixing temperature of 20-25 degree Celsius.

One or more water soluble fibers was premixed with the powdered one or more proteins in the low speed shear mixer for about 2 minutes, whereafter one or more texturizers was added including vegetable oil and lecithin emulsifier (if any) and mixed for about 3 minutes. Optional one or more sugar alcohols were also added with the one or more proteins after being pulverized. The temperature was slightly higher, if one or more sugar alcohols were added. Further texturizers (if any) including one or more of acacia, gelatine, carrageenan, and glycerin were then added and mixed for about 2 minutes. Hereafter, further ingredients were added, such as flavor, color, and optionally one or more active ingredients, and the mixture was blended until a total blending time of 10-12 minutes. If water was added, this was done after about 5 minutes of mixing, and in that case acacia and gelatine (if any) might preferably be premixed with the water at elevated temperature, whereas if one or more liquid or semi-liquid syrups was added, this was done at the same time as further texturizers, i.e., after about 5 minutes of mixing.

The mass of the inventive examples had a consistency of a soft chew tablet mass with visual appearance of an unspun coherent mass that was easy to transfer to other equipment for further processing into desired shapes.

The soft chew tablet mass obtained by low shear mixing (individual batches) was cooled to a temperature of about 20-30 degree Celsius, if needed due to heat formation during mixing, and formed into ropes by extrusion followed by cutting and forming into round-shaped soft chew cores of about 2-2.5 g per piece by means of a rotary forming machine. From a side view, the cores had an eclipse form. The temperature of cooling was critical in order to avoid problems in the formation of the soft chew tablet cores, particularly if one or more sugar alcohols were present.

### Example 2

### Formulation of Coated Soft Chew Tablets

Soft chew cores prepared according to Example 1 were optionally coated with a hard sugar free coating or chocolate coating after storage for about 12-48 hours.

In case of a hard coating, a suspension containing aqueous crystallizable sugar alcohol was applied onto the cores in a standard pan coating process if coating was applied, in which the water contents of the suspension applied was removed by blowing with air. This cycle was repeated 40-80 times in order to reach the swelling required, i.e., an increase in weight or thickness of the product.

A sub-coat was optionally applied around the soft chew cores in a pan coating process prior to applying the hard coating as outlined above. The pre-coat applied, if any, was performed with a blend in a specified ratio of Capol® 236 N provided by Capol GmbH, a brand of Freudenberg and powdered maltitol. The weight of the sub-coat was about 0.25-2% by weight of the soft chew tablet.

The hard coating, if applied, consisted of 65% by weight of maltitol crystals, 4% by weight of acacia, 30% by weight of unbound water and about 1% color and flavor. The total weight of the hard coating, if applied, was about 10-30% by weight of the soft chew tablet, the final weight of the hard coating depending directly on the number of coating cycles applied.

Following the same procedure as outlined above, other powdered sugars or powdered sugar alcohols may be used for the hard coating as an alternative to maltitol.

If a chocolate coating was applied as an outer coating, this was applied as one or more layers, depending on the thickness of the desired coating. A standard chocolate coating was applied as known to one skilled in the art.

### Example 3

### Formulation of Soft Chew Cores

Soft chew tablet cores with a weight of 2.2 g each were prepared according to Example 1.

TABLE 1
Raw material contents in %. Inulin was applied as Orafti HSI from Beneo.

| | B1 | B2 | B3 | B4 | B5 | B6 | B7 | B8 | C1 |
|---|---|---|---|---|---|---|---|---|---|
| Whey | 44.5 | 44.9 | 47.5 | 43.7 | 43.5 | 40.9 | 21.7 | 40.9 | 0 |
| permeate | | | | | | | | | |
| powder | | | | | | | | | |
| Hydrolyzed | 0 | 0 | 0 | 0 | 0 | 15 | 5 | 0 | 58.7 |
| protein | | | | | | | | | |
| powder | | | | | | | | | |
| Inulin | 40 | 40 | 40 | 40 | 40 | 25 | 55 | 40 | 0 |
| Maltitol | 0 | 0 | 0 | 0 | 0 | 0 | 0 | 6 | 38 |
| syrup | | | | | | | | | |
| Vegetable oil | 4 | 4 | 4 | 1 | 7 | 4 | 4 | 4 | 0 |
| Emulsifier | 0.3 | 0.3 | 0.3 | 0 | 0.3 | 0.3 | 0 | 0.3 | 0 |
| Gelatine | 1.8 | 1.8 | 1.8 | 2.7 | 1.8 | 1.8 | 1.3 | 1.8 | 0 |
| Acacia | 0.4 | 0 | 0.4 | 0.6 | 0.4 | 1 | 1 | 1 | 0 |
| Water | 6 | 3 | 0 | 6 | 0 | 6 | 6 | 0 | 0 |
| Glycerin | 0 | 3 | 3 | 3 | 4 | 3 | 3 | 3 | 0.3 |
| Cocoa | 3 | 3 | 3 | 3 | 3 | 3 | 3 | 3 | 3 |
| Total | 100 | 100 | 100 | 100 | 100 | 100 | 100 | 100 | 100 |

Soft chew tablet cores with a weight of 2.2 g each were prepared according to Example 1.

TABLE 2
Raw material contents in %. Inulin was applied as Orafti HSI from Beneo.
Hydrolyzed protein powder was applied as Nutrilac FO-8340 from Arla.

| | B11 | B12 | B13 | B14 | B15 | B16 | B17 | B18 |
|---|---|---|---|---|---|---|---|---|
| Hydrolyzed protein | 34.5 | 41.5 | 40.4 | 39.5 | 43 | 40.9 | 26.7 | 29 |
| powder | | | | | | | | |
| Inulin | 40 | 40 | 40 | 40 | 40 | 40 | 35 | 40 |
| Maltitol syrup | 7 | 0 | 0 | 0 | 0 | 0 | 19.8 | 12.5 |
| Vegetable oil | 4 | 4 | 4 | 4 | 2.7 | 4 | 4 | 4 |
| Emulsifier | 0.3 | 0.3 | 0.3 | 0.3 | 0.1 | 0.3 | 0.3 | 0.3 |
| Gelatine | 1.8 | 1.8 | 2.7 | 1.8 | 1.8 | 1.8 | 1.8 | 1.8 |
| Acacia | 0.4 | 0.4 | 0.6 | 0.4 | 0.4 | 1 | 0.4 | 0.4 |
| Water | 6 | 6 | 6 | 6 | 6 | 6 | 6 | 6 |
| Glycerin | 3 | 3 | 3 | 5 | 3 | 3 | 3 | 3 |
| Cocoa | 3 | 3 | 3 | 3 | 3 | 3 | 3 | 3 |
| Total | 100 | 100 | 100 | 100 | 100 | 100 | 100 | 100 |

Soft chew tablet cores with a weight of 2.2 g each were prepared according to Example 1.

TABLE 3
Raw material contents in %. Calcium carbonate grade Omyapure 35-OG was
obtained from Omya. Powdered sorbitol was prepared according to Example
1. Indigestible maltodextrin was applied as Fibersol-2 from ADM. Hydrolyzed
protein powder was applied as Nutrilac RM-7020 from Arla.

| | B20 | B21 | B22 | B23 | B24 | B25 | B26 | B27 |
|---|---|---|---|---|---|---|---|---|
| Hydrolyzed protein | 36.4 | 33.2 | 33.2 | 28 | 28 | 16.4 | 16.4 | 10.5 |
| powder | | | | | | | | |
| Indigestible | 40 | 36.3 | 36.3 | 30.2 | 30.2 | 34 | 34 | 38.6 |
| maltodextrin | | | | | | | | |
| Maltitol syrup | 0 | 0 | 2 | 0 | 2 | 0 | 2 | 0 |
| Vegetable oil | 4 | 4 | 2 | 4 | 2 | 4 | 2 | 4 |
| Emulsifier | 0.3 | 0.3 | 0.3 | 0.3 | 0.3 | 0.3 | 0.3 | 0.3 |
| Gelatine | 1.8 | 1.8 | 1.8 | 1.8 | 1.8 | 1.8 | 1.8 | 1.8 |
| Acacia | 1 | 1 | 1 | 1 | 1 | 1 | 1 | 1 |
| Water | 6 | 6 | 6 | 6 | 6 | 6 | 6 | 6 |
| Glycerin | 3 | 3 | 3 | 3 | 3 | 3 | 3 | 3 |
| Cocoa | 3 | 3 | 3 | 3 | 3 | 3 | 3 | 3 |
| Calcium carbonate | 4.5 | 11.4 | 11.4 | 22.7 | 22.7 | 30.5 | 30.5 | 31.8 |
| Total | 100 | 100 | 100 | 100 | 100 | 100 | 100 | 100 |

Soft chew tablet cores with a weight of 2.2 g each were prepared according to Example 1.

TABLE 4
Raw material contents in %. Calcium carbonate grade Omyapure 35-OG
was obtained from Omya. Powdered maltitol was prepared according
to Example 1. Inulin was applied as Orafti HSI from Beneo. Hydrolyzed
protein powder was applied as Nutrilac RM-7020 from Arla.

| | B30 | B31 | B32 | B33 | B34 | B35 | B36 | B37 |
|---|---|---|---|---|---|---|---|---|
| Hydrolyzed protein | 39.2 | 39.2 | 29.2 | 29.2 | 19.2 | 19.2 | 15.2 | 15.2 |
| powder | | | | | | | | |
| Inulin | 7.7 | 7.7 | 17.7 | 17.7 | 27.7 | 27.7 | 42.7 | 42.7 |
| Maltitol syrup | 0 | 2 | 0 | 2 | 0 | 2 | 0 | 2 |
| Vegetable oil | 4 | 2 | 4 | 2 | 4 | 2 | 4 | 2 |
| Emulsifier | 0.3 | 0.3 | 0.3 | 0.3 | 0.3 | 0.3 | 0.3 | 0.3 |
| Gelatine | 1.8 | 1.8 | 1.8 | 1.8 | 1.8 | 1.8 | 1.8 | 1.8 |
| Acacia | 1 | 1 | 1 | 1 | 1 | 1 | 1 | 1 |
| Water | 6 | 6 | 6 | 6 | 6 | 6 | 6 | 6 |
| Glycerin | 3 | 3 | 3 | 3 | 3 | 3 | 3 | 3 |
| Cocoa | 3 | 3 | 3 | 3 | 3 | 3 | 3 | 3 |
| Calcium carbonate | 34 | 34 | 34 | 34 | 34 | 34 | 23 | 23 |
| Total | 100 | 100 | 100 | 100 | 100 | 100 | 100 | 100 |

Soft chew tablet cores with a weight of 2.2 g each were prepared according to Example 1.

TABLE 4A
Raw material contents in %. Calcium carbonate grade
Omyapure 35-OG was obtained from Omya. Inulin was
applied as Orafti HSI from Beneo. Pea protein powder
was applied as Radipure E8001G from Cargill.

| | B40 | B41 | B42 | B43 | B44 | B45 |
|---|---|---|---|---|---|---|
| Whey permeate | 39 | 36 | 36 | 13 | 8 | 0 |
| powder | | | | | | |
| *Hydrolyzed | 4 | 10 | 10 | 0 | 5 | 0 |
| protein powder | | | | | | |
| Pea protein powder | 0 | 0 | 0 | 31 | 31 | 31 |
| Inulin | 38 | 35 | 35 | 27 | 17 | 17 |
| Glucose syrup | 0 | 0 | 0 | 0 | 20 | 20 |
| Vegetable oil | 4 | 4 | 4 | 4 | 4 | 4 |
| Emulsifier | 0.5 | 0.5 | 0.5 | 0.5 | 0.5 | 0.5 |
| Gelatine | 2 | 2 | 2 | 2 | 2 | 2 |
| Acacia | 0.5 | 0.5 | 0.5 | 0.5 | 0.5 | 0.5 |
| Water | 6 | 6 | 6 | 6 | 6 | 6 |
| Glycerin | 3 | 3 | 3 | 13 | 3 | 3 |
| Cocoa | 3 | 3 | 3 | 3 | 3 | 3 |
| Calcium carbonate | 0 | 0 | 0 | 0 | 0 | 13 |
| Total | 100 | 100 | 100 | 100 | 100 | 100 |

*Two different kinds of hydrolyzed protein powders applied in samples B41 and B42.

Relevant for all inventive samples above, the cores that were provided with a hard coating or chocolate according to Example 2 revealed surprisingly advantageous properties as a partial moisture barrier to ingredients in the core of the soft chew tablet.

Additionally, relevant for all inventive samples above, the cores that were provided with a hard coating or chocolate revealed surprisingly advantageous properties as a partial anti-sticking and moisture barrier to ingredients in the core of the soft chew tablet.

Compared to cores with a sugar content instead of a protein powder, the inventive samples without a coating showed surprising improvements in terms of less stickiness.

### Example 4

### Sensorial Evaluation of Coated Soft Chew Tablets

The soft chew cores of Example 3 were evaluated after application of coating according to Example 2.

For evaluation a panel of 7 trained assessors was used. Each assessor repeated the evaluations twice.

The trained assessors abstain from eating and drinking at least 30 minutes before initiation of any test. Each trained assessor was a healthy person appointed on an objective basis according to specified requirements. The sensory analysis was performed according to ISO 4121-2003 in testing conditions following ISO 8589. The result is an average of the results of the 7 individuals.

The test persons gave a rating from “+” to “+++++”, where “+” is poor and “+++++” is excellent.

Rating “+++” means that the tablet was comparable to the standard, whereas “+” means that the tablet was very far from comparable to the standard and “+++++” means that the tablet is exceeding the rating of the standard. “0” indicated that it was not tested.

Different parameters were tested in a test panel:

| | | | |
|---|---|---|---|
| Mouthfeel | Tackiness | Softness | Off-notes |

“Mouthfeel”—the general impression of the tablet when placed in the mouth with respect to elements such as cohesiveness, elasticity, and texture.
“Tackiness” the ability of the tablet to stick to teeth during chewing. A soft chew tablet should possess some tackiness but neither too little or too much. I.e. a score of “+++” is considered ideal for this parameter
“Softness”—the softness of the tablet. Both a slightly softer and a slightly harder than average tablet would be considered a good soft chew tablet
“Off-notes”—the overall impression of the off-note from the one or more active ingredients in the composition during chewing. For instance, if off-notes (chalkiness, bitter notes, irritation in the throat) were experienced, a low rating was given and if other uncomfortable sensations were experienced, a low rating was also given.

### Example 5

### Qualitative Sensorial Evaluation of Chocolate Coated Soft Chew Tablets

A qualitative sensorial evaluation was made of the soft chew cores of Example 3 after application of a chocolate coating according to Example 2.

| | Total sensory | |
|---|---|---|
| | experience: | |
| | Very good | |
| | Good | |
| | Acceptable(Acc) | |
| | Poor | |
| Sample | Very poor | Comments |
| C1 | Very poor | Very soft chewing texture, very little |
| | | chewing resistance, poor tackiness |
| B40 | Very good | Slightly firm, coherent chewing texture, very |
| | | good chewing resistance, good “just right” |
| | | tackiness |
| B41 | Very good | Slightly firm, coherent chewing texture, very |
| | | good chewing resistance, good “just right” |
| | | tackiness |
| B42 | Very good | Slightly firm, coherent chewing texture, very |
| | | good chewing resistance, good “just right” |
| | | tackiness |
| B44 | Good | Slightly firm, coherent chewing texture, |
| | | good chewing resistance, slightly high |
| | | tackiness |

### Example 6

### Hardness Tests

Various samples of representative soft chew cores were tested on a standard texture analyzer in order to measure the hardness of selected soft chew cores before a coating was applied according to Example 2 was provided. The texture analyzer was provided by Stable Micro Systems under the name TAXT2i Texture Analyser. The texture analyzer was equipped with a needle that was applied to the top surface of the cores. The force applied to penetrate the cores was measured in Newton (N).

TABLE 8
Measured on standard texturizer during storage over time.

| Hardness (N) | B40 | B41 | B42 |
|---|---|---|---|
| 2 days | 15 | 20 | 14 |
| 6 days | 17 | 23 | 17 |
| 10 days | 18 | 27 | 20 |

As can be seen from the tests of hardness, various of the samples had a superior hardness already after a few days of storage. Relevant for B40 to B42, it was surprisingly experienced that a superior mouthfeel and texture was present and that the sample resembled soft chew tablet, despite the powdered sugar components of the standard soft chew tablet being replaced with powdered proteins in the inventive samples. Surprisingly, the hardness during storage was found relatively stable and did not necessitate an outer coating.

1. A soft chew tablet based on proteins, the soft chew tablet comprising:
a core constituting a coherent, uncooked and extruded mass comprising:
one or more indigestible water soluble fibers in an amount of 10 to 45% by weight of the core,
one or more powdered proteins in an amount of 10 to 60% by weight of the core, and
one or more texturizers in an amount of 0.1 to 25% by weight of the core.

2. (canceled)

3. (canceled)

4. The soft chew tablet according to claim 1, wherein the one or more powdered proteins has been preserved during formation of the core.

5. (canceled)

6. (canceled)

7. The soft chew tablet according to claim 1, wherein the coherent, uncooked and extruded mass is a coherent mass with remains of particles.

8. The soft chew tablet according to claim 1, wherein the coherent, uncooked and extruded mass comprises sugar and/or sugar alcohol.

9. The soft chew tablet according to claim 1, wherein the coherent, uncooked and extruded mass is a coherent mass with remains of sugar particles and/or sugar alcohol particles.

10. The soft chew tablet according to claim 1, wherein the coherent, uncooked and extruded mass is characterized by a texture resembling a soft chew tablet mass with remains of particles that does not crumble upon chewing.

11.-17. (canceled)

18. The soft chew tablet according to claim 1, wherein the coherent, uncooked and extruded mass is formed at a temperature below 60 degree Celsius.

19.-26. (canceled)

27. The soft chew tablet according to claim 1, wherein the one or more indigestible water soluble fibers has a Dextrose Equivalent (DE) of 3 to 20.

28. The soft chew tablet according to claim 1, wherein the one or more indigestible water soluble fibers is selected from the group consisting of oligosaccharides, indigestible carbohydrate fibers, polydextrose, inulin, indigestible maltodextrin, and any combination thereof.

29.-32. (canceled)

33. The soft chew tablet according to claim 1, wherein the one or more indigestible water soluble fibers comprises at most 0.5% sugars by weight of the one or more indigestible water soluble fibers.

34.-39. (canceled)

40. The soft chew tablet according to claim 1, wherein the core further comprises one or more liquid or semi-liquid syrups in an amount of 0.1 to 30% by weight of the core.

41. (canceled)

42. (canceled)

43. The soft chew tablet according to claim 1, wherein the core further comprises one or more liquid or semi-liquid sugar alcohol syrups selected from the group consisting of maltitol syrup, sugar based syrups, sugar based syrups comprising glucose, and any combination thereof.

44.-46. (canceled)

47. The soft chew tablet according to claim 1, wherein the core further comprises cocoa in an amount of 1 to 7% by weight of the core.

48.-52. (canceled)

53. The soft chew tablet according to claim 1, wherein the one or more powdered proteins is present in an amount of 20 to 45% by weight of the core.

54.-56. (canceled)

57. The soft chew tablet according to claim 1, wherein the one or more powdered proteins replaces powdered sugars or powdered sugar alcohols.

58. (canceled)

59. (canceled)

60. The soft chew tablet according to claim 1, wherein the one or more powdered proteins comprises isolated, concentrated or hydrolyzed dairy proteins.

61. The soft chew tablet according to claim 1, wherein the one or more powdered proteins comprises proteins selected from the group consisting of proteins originating from casein, calcium caseinate, whey, and combinations thereof.

62. (canceled)

63. (canceled)

64. The soft chew tablet according to claim 1, wherein the one or more powdered proteins comprises isolated, concentrated or hydrolyzed plant proteins.

65. The soft chew tablet according to claim 1, wherein the one or more powdered proteins comprises plant proteins selected from the group consisting of proteins originating from pea, soy, faba beans, chia, rice, oat, wheat, potato, canola, sunflower, rapeseed, and combinations thereof.

66. (canceled)

67. (canceled)

68. The soft chew tablet according to claim 1, wherein the core further comprises one or more powdered sugar alcohols present in an amount of 5 to 40% by weight of the core.

69.-73. (canceled)

74. The soft chew tablet according to claim 1, wherein the core further comprises one or more powdered sugar alcohols with an average particle size of less than 100 microns.

75.-79. (canceled)

80. The soft chew tablet according to claim 1, wherein the core further comprises calcium carbonate in an amount of 5-20% by weight of the core.

81.-86. (canceled)

87. The soft chew tablet according to claim 1, wherein the one or more texturizers is present in an amount of 0.5 to 20% by weight of the core.

88.-95. (canceled)

96. The soft chew tablet according to claim 1, wherein the one or more texturizers comprises one or more of lecithin, gelatine, carrageenan, instant starch, acacia, or glycerin.

97.-103. (canceled)

104. The soft chew tablet according to claim 1, wherein the one or more texturizers comprises a vegetable oil, lecithin, gelatine, acacia, and glycerin.

105. The soft chew tablet according to claim 1, wherein the core further comprises one or more active ingredients present in an amount of 1 to 60% by weight of the core.

106.-110. (canceled)

111. The soft chew tablet according to claim 1, wherein the core further comprises one or more active ingredients selected from the group consisting of one or more electrolytic active ingredients, one or more energy stimulating active ingredients, one or more vitamins, one or more minerals, and any combination thereof.

112.-129. (canceled)

130. The soft chew tablet according to claim 1, wherein the soft chew tablet comprises an outer coating comprising a chocolate coating.

131.-143. (canceled)
